(19) United States
(12) Patent Application Publication
(10) Pub. No.: US 2025/0072935 A1
(43) Pub. Date: Mar. 6, 2025

(54) VISUAL EMBRYO TRANSFER SYSTEM, KITS AND METHODS OF USE

(71) Applicant: BUTTERFLY BIOSCIENCES, INC., Oakland, CA (US)

(72) Inventors: Moses CESARIO, Oakland, CA (US); Alexander NADAL, Los Angeles, CA (US); Sam NAJMABADI, Beverly Hills, CA (US); Steven NAKAJIMA, Broomfield, CO (US); Ricardo PERAZA, Bucerias (MX); José Luis RIVAS, Punta de Mita (MX)

(21) Appl. No.: 18/766,266

(22) Filed: Jul. 8, 2024

Related U.S. Application Data
(60) Provisional application No. 63/525,537, filed on Jul. 7, 2023.

(51) Int. Cl. A61B 17/435; A61B 1/015; A61B 1/303; A61B 17/42; A61B 90/00; G16H 10/60; G16H 30/20

(52) U.S. Cl. CPC A61B 17/435; A61B 1/015; A61B 1/303; A61B 90/361; A61B 90/37; G16H 10/60; G16H 30/20; A61B 2017/4225

(57) ABSTRACT

A visual embryo transfer catheter system includes a handle and a visualization sheath which is configured to be coupled to the handle and to be inserted into the uterus of a patient. The visualization sheath includes at least three lumens, including a first lumen for a scope assembly including a camera and an illumination source, a second lumen to permit passage of an embryo transfer catheter which is configured to deliver an embryo to the uterus upon imaging of the uterus with the camera when the uterus is illuminated by the illumination source, and a third fluid supply lumen for inflow and outflow of fluid to and from the uterus. Further embodiments include methods for transferring an embryo into a uterus of a patient under visual guidance, and kits for performing a visualized embryo transfer.

### RELATED APPLICATION

This application claims the benefit of priority to U.S. Provisional Application No. 63/525,337, entitled “Visual Embryo Transfer System, Kits and Methods of Use,” filed on Jul. 7, 2023, the entire contents of which are incorporated by reference herein for all purposes.

### TECHNICAL FIELD

The present invention relates generally to the field of Assisted Reproductive Technology (ART) and, more specifically, embryo transfer.

### BACKGROUND

Embryo transfer (ET) is a critical step in the in vitro fertilization (IVF) process, wherein a fertilized embryo is transferred into the uterus of a woman with the goal of achieving a successful pregnancy. It is the last step in the IVF process, and the crucial procedure in which the work of several weeks of the IVF process culminates. The transfer must be carried out with high precision and with minimal complication to increase the probability of embryo implantation.

The conventional method of embryo transfer involves the use of a soft catheter and a rigid outer sheath. The rigid outer sheath houses the soft embryo transfer catheter and the distal end of the outer sheath is positioned at the internal cervical ostium. The soft catheter is then placed through the outer sheath and extends beyond the outer sheath into the uterus. The tip of the embryo transfer catheter is positioned either blindly (e.g “clinical touch”) or under ultrasound guidance. The embryo is then expelled from the catheter and deposited within the uterine cavity.

Three factors determine success of an embryo transfer: 1) quality of the embryo, 2) endometrial receptivity, and 3) the quality of the embryo transfer procedure. The quality of the procedure is affected by several factors, including the use of ultrasound to guide the tip of the catheter, the presence of mucus and blood, the position and anatomy of the uterus, cervical stenosis, retention of the embryo in the catheter, clinical experience and the correct positioning of the tip of the catheter inside the uterus (Alvero et al., 2003; Arora & Mishra, 2018; Kava-Braverman et al., 2017; Mains & Van Voorhis, 2010; Plowden et al., 2017; Singh et al., 2020; Tiras et al., 2012). Taken together the foregoing factors determine whether a transfer is “easy” or “difficult.” Anywhere between 5% to 15% of embryo transfers are considered difficult.

Several studies indicate that the degree of difficulty during embryo transfer is correlated with implantation and pregnancy rates; when transfers are difficult, pregnancy rates are reduced by 5% to 10% (Ghaffari et al., 2013; Kava-Braverman et al., 2017; Listijono et al., 2013; Tomás et al., 2002). In a study from 2017, Kava-Braverman and colleagues analyzed 7,714 embryo transfers and observed that clinical pregnancy rates are higher in the cases of easy embryo transfer compared with difficult embryo transfers (38.2% vs 27.1%). The use of additional instrumentation during embryo placement, such as a stylet, tenaculum, hysterometer, and outer catheter in the fundus, correlates with a progressive reduction in the clinical pregnancy rate. Similarly, Listijono and collaborators in 2013 found statistically significant differences in the live delivery rates (25% vs 19.5%, p<0.05), clinical pregnancy rates (30.7% vs 24.6%, p<0.5) between the easy and medium or difficult groups. Likewise, Ghaffari et al analyzed 706 embryo transfers and found a significantly higher implantation rate in the easy group (21.7%) compared to the difficult group (12.1%, p<0.05). The easy group had a higher pregnancy rate (38.1%) compared to patients who had difficult embryo transfers (21.4%, p<0.05).

A primary contributor to a difficult embryo transfer is the anatomical variation among patients. An easy transfer is likely to occur when the uterus has normal anatomy (e.g straight and/or slightly anteverted) and allows the passage of the catheter quickly and without impediments through the cervix to the uterine cavity. Where the anatomy is atypical, the procedure is likely to be referred to as a difficult transfer. Common atypicalities include: endocervical crypts, tortuous cervical canal, uterine anteversion, cesarean induced isthmocele, false pathways, stenosis of the internal and external cervical ostium, malformation, conization, cervical endometriosis and poor cervical exposure. And, when a cause for difficult embryo transfer exists, 53% have two or more causes (Larue, 2020).

The outcome of an embryo transfer is heavily influenced by the operator's ability to deposit the embryos at the most suitable location for implantation, without causing harm to the endometrium (Coroleu et al. 2002). Moreover, avoiding the induction of uterine contractions (Fanchin et al. 1998), and preventing any iatrogenic damage to the embryos are essential for achieving successful results. The research consistently shows that the operator performing ET significantly impacts the ongoing pregnancy rate (OPR) in assisted reproduction (Cirillo et al., 2020, 2022, 2019; Mizrachi & McQueen, 2022). Studies spanning over 20 years and involving tens of thousands of ETs demonstrate significant heterogeneity in success rates among operators, with odds ratios ranging from 0.84 to 1.13 compared to the mean (Cirillo et al., 2020). Interestingly, experience does not necessarily correlate with improved outcomes, as no significant association was found between an operator's experience and OPR (Cirillo et al., 2022, 2019). Both physicians and embryologists performing ET influence outcomes, with physicians having a slightly greater impact (Cirillo et al., 2022). These studies collectively highlight the importance of standardized, atraumatic transfer techniques in equalizing success rates among physicians and potentially improving overall outcomes in assisted reproductive technology procedures.

In summary, the limitations of the current state of the art of an embryo transfer include insufficient image resolution due to the limitations inherent in ultrasound, difficulty traversing atypical anatomies, difficulty in identifying the exact location to transfer the embryo, and over-reliance on the operator's skill and experience. As global IVF cycles expand in the wake of automation and standardization of the IVF process, there is a need for an improved embryo transfer catheter that provides more accurate positioning of the embryo within the uterine cavity by using direct visualization, ultimately increasing the success rate of IVF procedures.

### SUMMARY

The present invention describes a visual embryo transfer system representing a significant advancement for the field of Assisted Reproductive Technologies (ART). Since the 1980's, embryo transfer has utilized a stiff outer catheter guide and a soft, flexible transfer catheter which are placed into the uterus either blindly or under ultrasound guidance to deliver an embryo into a uterus.

The Visual Embryo Transfer (VET) System 90 consists of 1) a Visualization Sheath 180, and 2) an Embryo Transfer Catheter 100 (ET Catheter). The Visualization Sheath 180 is a steerable, multi lumen sheath with an integrated camera system that provides real time view of the internal anatomy of the endometrial cavity. The Embryo Transfer Catheter 100 is a single lumen soft catheter which is used to carry the embryo into the uterus. Novel methods of use are described herein for performing embryo transfer with the VET system.

The key features in the VET System include the CMOS “chip on tip” camera and illumination provided by glass fibers; a Scope Assembly 290 which bundles the CMOS camera 280 and illumination fibers 300; real time visualization of the procedure displayed on an onboard Touchscreen Display 190, or external monitor; steerability controlled by the user; and a multi-lumen Visualization Sheath 180 which conducts the Embryo Transfer Catheter 100, the Scope Assembly 290, and at least one or more lumens for fluid delivery and collection 330. Use of the VET System uses clear media for insufflation of the uterus with a small volume of fluid media to view the inside of the cavity and appropriately position the tip of the ET Catheter. Any residual fluid is removed from the uterus prior to expulsion of the embryo into the uterus.

In one embodiment a visual embryo transfer system is provided which includes a handle including a Video Display 190 for displaying the interior of a uterus of a patient upon imaging of the uterus with a camera; a scope assembly comprising a camera and an illumination source; an Embryo Transfer Catheter 100 which is configured to deliver an embryo to the uterus upon imaging of the uterus with the camera when the uterus is illuminated by the light source; and a Visualization Sheath 180 which is configured to be coupled to the handle and to be inserted into the uterus of a patient, the sheath comprising at least three lumens, a first lumen 270 for the Scope Assembly 290, a second lumen 340 to permit passage of the Embryo Transfer Catheter 100 therethrough, and a third fluid supply lumen 330 for inflow and outflow of fluid to and from the uterus.

The camera of the VET system 90 may comprise a CMOS camera 280. The illumination source may comprise illumination fibers 300 that are concentrically distributed around the CMOS camera to evenly distribute light within the uterus. The illumination source may comprise light wavelengths of greater than approximately 550 nm to avoid damage to an embryo placed into the uterus. The handle may include internal hardware including an image processing board, at least one microcontroller, a rechargeable battery, a wireless connectivity unit. The handle is configured to reusably couple to the Visualization Sheath 180. A wireless connectivity unit in the handle is used to create a secure HIPAA compliant connection to an online software-as-a-service (Saas) application. The SaaS application captures patient data that details the visual embryo transfer procedure. The patient data is placed into a physician portal to become available for a physician office to access the data.

In another embodiment a method for visual embryo transfer into a uterus of a patient is provided which comprises providing a handle including a Video Display 190 for displaying the interior of a uterus of a patient upon imaging of the uterus with a camera; and a Visualization Sheath 180 which is configured to be coupled to the handle and to be inserted into the uterus of the patient, the sheath comprising at least three lumens, a first lumen 270 for the Scope Assembly 290 which includes a camera and an illumination source, a second lumen 340 to permit passage of the Embryo Transfer Catheter 100 therethrough, and a third fluid supply lumen 330 for inflow and outflow of fluid to and from the uterus; inserting the Embryo Transfer Catheter 100 into the Visualization Sheath 180; inserting the Visualization Sheath 180 into the uterus; supplying fluid to the third fluid supply lumen 330 to insufflate the uterus with fluid; advancing the Embryo Transfer Catheter 100 into the uterus and positioning the tip of the catheter over an optimal location for embryo transfer; removing all fluid from the uterus with negative pressure; transferring an embryo into the uterus by depressing a Syringe 160 on the Embryo Transfer Catheter 100; and removing the Visualization Sheath 180 from the uterus. The method further comprises illuminating a portion of the uterus to be imaged with a light source and imaging the uterus that is illuminated with the camera. The method may comprise delivering approximately 1 cc to 10 cc of fluid to the uterus via the third fluid supply lumen 330 of the Visualization Sheath 180.

In another embodiment, a method for transferring an embryo into a uterus of a patient under visual guidance is disclosed which comprises supplying fluid to the uterus of the patient to insufflate the uterus; illuminating a portion of the uterus with a light source and imaging the uterus that is illuminated with a camera; removing all fluid from the uterus following insufflation of the uterus; inserting an Embryo Transfer Catheter 100 into the uterus and positioning a tip of the catheter over an optimal location for embryo transfer; and transferring an embryo into the uterus under visual guidance from the camera. The supplying fluid may comprise delivering approximately 1 cc to 10 cc of fluid to the uterus. The removing fluid may comprise using negative pressure to remove the fluid. The illuminating a portion of the uterus may comprise illuminating the portion with a wavelength greater than approximately 550 nm. The method may further comprise capturing of patient data that details the visual embryo transfer method, wherein the patient data can be placed into a physician portal to become available for a physician office to access the patient data. Other embodiments and methods of use are also envisioned and are described herein.

In another embodiment, the VET unit is provided as part of an embryo transfer management system comprising a handle assembly including a video display for displaying the interior of a uterus of a patient upon imaging of the uterus with a camera; a holder that secures the handheld assembly at the top of the system; a scope assembly comprising a camera and an illumination source; an embryo transfer catheter which is configured to deliver an embryo to the uterus upon imaging of the uterus with the camera when the uterus is illuminated by the light source; a programmable pump operably coupled to the embryo transfer catheter to provide a controlled release of an embryo into the uterus of the patient; a visualization sheath which is configured to be coupled to the handle assembly and to be inserted into the uterus of a patient, the sheath comprising at least three lumens, a first lumen for the scope assembly, a second lumen to permit passage of the embryo transfer catheter therethrough, and a third fluid supply lumen for inflow and outflow of fluid to and from the uterus; and a peristaltic pump and a vacuum pump operably coupled to the third fluid supply lumen to automatically provide fluid to the uterus via the fluid supply lumen and/or to remove fluid from the uterus. The visualization sheath may also comprise one or more additional lumens, e.g., a dedicated fluid supply lumen to remove fluid from the uterus, e.g., upon application of suction from a vacuum source.

The embryo transfer management system may further comprise a foot pedal operably coupled to the programmable pump. The programmable pump can also be controlled via the video display on the handheld assembly. In one exemplary embodiment, the programmable pump is programmed to have a fluid infusion rate of about 5-15 μl/s for about 2-3 seconds. The embryo transfer management system may further comprise at least one pressure sensor which is capable of monitoring intrauterine pressure. The pressure sensor may be operably coupled to the peristaltic pump and vacuum pump which in one embodiment may be controlled to maintain a target pressure range of between about 5 mmHg to 50 mmHg in the uterus. In one embodiment, the pressure pump is programmed to provide a fluid inflow rate at about 0.6 mL/s. In one embodiment, the vacuum pump is programmed to provide suction at a fluid rate of above 2 ml/s with a suction pressure of about 100 mmHg.

In another embodiment of the invention, a method for visual embryo transfer into a uterus of a patient comprising: using a handle including a video display for displaying the interior of a uterus of a patient upon imaging of the uterus with a camera; and a visualization sheath which is configured to be coupled to the handle and to be inserted into the uterus of the patient, the sheath comprising at least three lumens, a first lumen for a scope assembly which scope assembly includes a camera and an illumination source, a second lumen to permit passage of the embryo transfer catheter therethrough, and a third fluid supply lumen for inflow and outflow of fluid to and from the uterus; inserting the embryo transfer catheter into the visualization sheath; inserting the visualization sheath into the uterus; automatically supplying fluid to the third fluid supply lumen to insufflate the uterus with fluid using a programmable pressure pump; advancing the embryo transfer catheter into the uterus and positioning the tip of the catheter over an optimal location for embryo transfer; automatically removing all fluid from the uterus with negative suction pressure from a programmable vacuum source; automatically transferring an embryo into the uterus using a programmable pump operably coupled to the embryo transfer catheter to provide a controlled release of an embryo into the uterus of the patient; and removing the visualization sheath from the uterus. The visualization sheath may also comprise one or more additional lumens, e.g., a dedicated fluid supply lumen to remove fluid from the uterus, e.g., upon application of suction from the programmable vacuum source.

In one embodiment, the programmable pump is programmed to have a fluid infusion rate of about 5-15 μl/s for 2-3 seconds. In one embodiment, the supplying fluid comprises delivering approximately 1 cc to 10 cc of fluid to the uterus. In one embodiment, the programmable pressure pump is programmed to supply fluid at a fluid flow rate of about 0.6 mL/s and the vacuum pump is programmed to provide suction at a rate of about 2 ml/s with a suction pressure of about 100 mmHg. The removing fluid may comprise using negative pressure to remove the fluid from a fourth lumen in the visualization sheath. The illuminating a portion of the uterus may comprise illuminating the portion with light in a range of wavelengths greater than approximately 550 nm. In one embodiment, the programmable pump is activated via a foot pedal or alternatively the programmable pump is activated via the video display. The method may further comprise removing air from the embryo transfer catheter prior to inserting an embryo into the catheter.

In another embodiment, a kit for performing a visual embryo transfer procedure is provided which includes a reusable handle assembly; an embryo transfer catheter which is configured to deliver an embryo to the uterus; a visualization sheath which is configured to be coupled to the handle assembly and to be inserted into the uterus of a patient, the sheath comprising at least three lumens, a first lumen for insertion of scope assembly into the uterus comprising a camera and a light source, a second lumen to permit passage of the embryo transfer catheter therethrough, and a third fluid supply lumen for inflow of fluid to and/or from the uterus; and an embryo transfer culture medium. The kit may further comprise a syringe. The kit may further include an hCG wash fluid for the uterus.

The VET System combines new technological features and methods of use to significantly improve embryo transfer by facilitating navigation of the VET System through the cervix and into the uterus via steerability, and real time visualization to ensure the most precise placement of the embryo in the uterine cavity.

### BRIEF DESCRIPTION OF THE DRAWINGS

FIG. 1 shows a fully assembled VET System 90 with the Embryo Transfer Catheter 100 placed into the Visualization Sheath 180 and extending beyond the Visualization Sheath Tip 110.

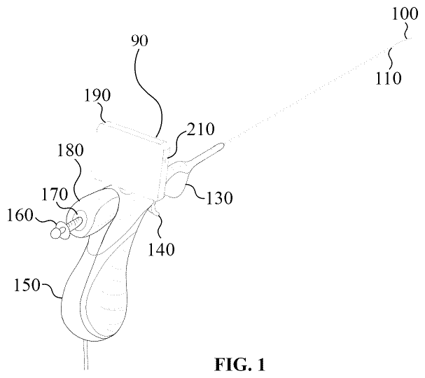

FIGS. 2A and 2B show the reusable handle. FIG. 2A shows the right face and FIG. 2B shows the left face which is the side that interfaces electronically and mechanically with the Visualization Sheath 180.

FIGS. 3A and 3B show the main body of the Visualization Sheath 180. FIG. 3A shows the right face of the body which interfaces electronically and mechanically with the Visualization Sheath 180. FIG. 3B shows the left face of the Visualization Sheath 180 body.

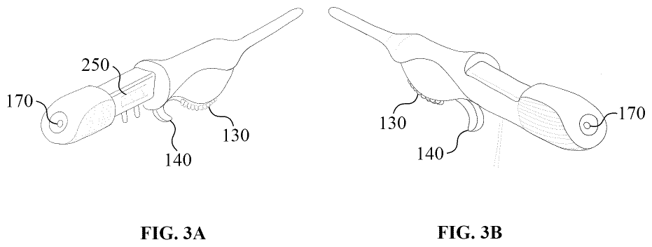

FIGS. 4A and 4B show the Visualization Sheath Tip 110. This is the area which traverses the cervix and is placed into the uterus. FIG. 4A shows a close up of the distal tip, FIG. 4B shows a close up of the distal tip with the Embryo Transfer Catheter 100 inserted into the dedicated lumen 340.

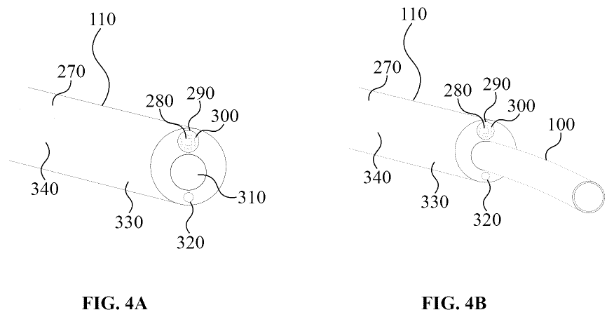

FIG. 5 shows the Embryo Transfer Catheter 100.

FIG. 6 shows the steerability function of the Visualization Sheath 180.

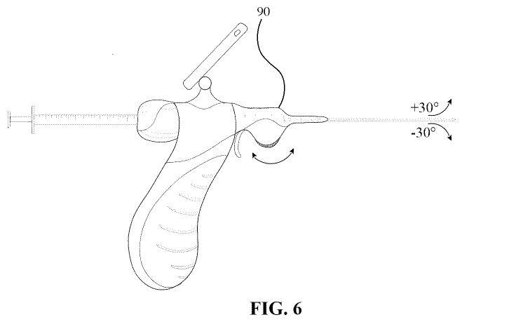

FIGS. 7A, 7B, and 7C show the Embryo Transfer Management System (ETMS) from different perspectives. FIG. 7A provides a face on perspective, FIG. 7B provides a side view, and FIG. 7C provides an angled view.

FIGS. 8A and 8B show the ETMS version of the reusable handle at different perspectives. FIG. 8A provides an angular view, and FIG. 8B provides a side view.

FIG. 9 shows the ETMS version of the reusable handle with a direct view of the steerable shaft 500, and a close-up of the shaft tip showing the CMOS camera 280, and the illumination light fibers 300.

FIG. 10 shows a side view of the ETMS version of the disposable Visualization Sheath 180.

FIG. 11A shows an angular side view of the ETMS version of the disposable Visualization Sheath 180, and FIG. 11B shows a close up of the multi-lumen visualization sheath tip 110.

FIG. 12 shows the full ETMS version of the visual embryo transfer handheld assembly 560 from a side view.

FIGS. 13A and 13B show two perspectives of the ETMS version of the visual embryo transfer handheld assembly 560. FIG. 13A provides a rear-facing angular view, and FIG. 13B provides a front-facing angular view.

FIG. 14 shows the optimal embryo loading technique for visual embryo transfer, and the conventional embryo loading technique.

### DETAILED DESCRIPTION OF THE INVENTION

### Overview and Components

Visual Embryo Transfer (VET) is performed to place a single or multiple embryos into the uterus of a woman and establish pregnancy and/or capture a biopsy of the uterine space at the time of embryo transfer.

The VET procedure is done within the window of implantation, approximately 8-10 days after ovulation.

The optimal configuration of the visual embryo transfer system includes a Visualization Sheath 180 comprised of the following components: 1) CMOS camera 280, 2) illumination source and fibers 300, 3) tip steerability, and 4) multiple lumens which house the Scope Assembly 290, conduct the Embryo Transfer Catheter 100, and conduct fluid inflow and outflow. The Visualization Sheath 180 is compatible with a specialized Embryo Transfer Catheter 100 with a stainless steel stiffening rod 360 that is placed into the Visualization Sheath 180 via a seal at the proximal end of the Visualization Sheath 180. The Embryo Transfer Catheter 100 is a soft catheter device which is placed into the dedicated ET Catheter lumen 340 of the Visualization Sheath 180 and can extend beyond the distal end of the Visualization Sheath 180. The Visualization Sheath 180 interfaces with a handle component which performs image processing, real time video display, capture of data with local storage SaaS-based data-based connectivity through secure HIPAA compliant wireless connectivity.

The placement of the Embryo Transfer Catheter 100 prior to transfer of the embryo(s) is achieved utilizing the steering apparatus, the camera placed at Visualization Sheath Tip 110, the light source, and fluid insufflation of the uterus. The physician positions the Visualization Sheath Tip 110 in line with the internal cervical ostium using the steering apparatus in conjunction with the camera and fluid insufflation to allow for visualization to enter the cervix via the external cervical ostium, navigate the cervical canal using steering controls, enter the internal ostium, insufflate with fluid to gain visualization of the uterus, insertion and of the ET catheter into the desired location within the uterus. With the catheter placed, the insufflation fluid is removed and finally, the embryo is transferred.

Two methods can be used for embryo transfer depending upon physician preference: preload or afterload. Those methods are described herein.

The Visualization Sheath 180 and catheter work together to improve the process of embryo transfer in the following ways.

Ability to Place Embryo Transfer Catheter and Embryo accurately: A successful embryo transfer is achieved when the embryo(s) are placed at the center of the uterine cavity, equidistant from the fundus and the internal ostium, equidistant from each medial edge of the uterine cavity, and in between the two endometrial layers which have formed on the anterior and posterior aspects of the cavity or at a place or location of the physicians' choosing based upon any unique aspects of the physiology of the particular patient. Using a standard ET catheter (current standard of care) in up to 15% of embryo transfer cases the anatomy including the cervical canal, vagina and uterus, or poor ultrasound visualization may cause the physician to be uncertain of placement (i.e. a difficult embryo transfer) (Ghaffari, 2013). Direct visualization coupled with steerability overcomes the challenges that may be present during the embryo transfer. When the embryo is transferred the physician knows with certainty that the transfer catheter has been placed in the desired position.

Visualization as an improved method for placement confirmation: The VET catheter uses direct visualization instead of relying solely on ultrasound for placement of the transfer catheter. Ultrasound provides a planar, one-dimensional view of the anatomy that requires skill by physician to interpret the ultrasound image and skill by the ultrasound technician in correct placement of the ultrasound probe during the embryo transfer and in many cases ultrasound visualization may be impacted by high BMI, or physiologic anomaly, or uterine position. Direct visualization is superior because it provides an accurate view of the whole uterine cavity reliably regardless patient variation. Direct visual view of the anatomy during the embryo transfer procedure is also intuitive and hence obviates the need for high physician skill by simplifying the overall transfer process. Complexity is further reduced because it does not require an ultrasound technician, assistance by a nurse or other actions that require a high degree of coordination by the physician to execute the procedure.

Reduction of embryo manipulation time: If placement of an Embryo Transfer Catheter 100 takes excessive time due to initial placement of the catheter through the cervical canal, or due to uterine position (antiflex/retroflex, anteverted/retroverted) the placement is at an angle medial to or distal to the uterine canal, the process of embryo transfer may prolong the exposure of the embryo to the catheter and sustain it in a state of manipulation for excess time. This manipulation may impact the process of embryo attachment at the endometrium.

Confirmation of placement of an embryo between the endometrial layers with a minimum of disturbance to the endometrium: If an Embryo Transfer Catheter 100 is positioned at an angle that is not along the midline of the uterus, when the Embryo Transfer Catheter 100 is extended into the uterine space, the Embryo Transfer Catheter 100 may tunnel into either the anterior or posterior endometrial layers such that the embryo may be transferred at a location not in between the endometrial layers that have formed. Under the current standard of care, which relies on ultrasound guidance, an incorrect placement is possible because the ultrasound view of the uterus is planar and does not provide enough anatomical detail to ensure that the correct placement may occur.

Patient discomfort during transfer: Providing steerability and visualization during the embryo transfer procedure minimizes the need to readjust the angle of the Embryo Transfer Catheter 100 and minimizes the need to adjust the anatomy during catheter placement. If the patient presents with a tortuous cervical canal, a uterus in a location significantly deviated from the midline of the uterine canal/vagina, or a uterus with flexion, the physician may utilize bladder pressure and or palpation to straighten the uterus which extends the time period the patient remains in the viewing room, and may cause patient discomfort and uterine cramping prior to the procedure. Steerability and visualization obviate these common issues experienced during placement and transfer.

Reducing uterine contractions: The VET catheter reduces uterine manipulation during embryo transfer procedures, thereby minimizing patient discomfort and the incidence of uterine contractions. The VET catheter's real-time visualization and maneuverability allow the catheter to be introduced and positioned within the uterus, thus reducing the need for additional instruments to adjust the uterine angle. This capability to steer in response to the uterine structure ensures precise placement of the embryo with minimal intervention. Excessive uterine manipulation, as well as accidentally touching the fundus of the uterus, can lead to uterine contractions, which are detrimental to embryo implantation (Chung et al., 2017; Fanchin et al., 1998; Javedani et al., 2023; Zhu et al., 2014). By mitigating these factors, the VET catheter enhances the likelihood of successful embryo implantation and improves patient comfort during the procedure.

Visualization may eliminate need for ultrasound: Ultrasound is the standard of care for use in embryo transfer however it is imprecise and ineffective in cases where a patient's uterus is significantly retroverted or anteverted or the patient has a high BMI (Russo, M et. al)¹. This statistic may be partially caused by the inability to utilize ultrasound to ensure correct placement of the embryo during en ET procedure. With visualization the need for ultrasound is obviated and hence possesses the potential to increase pregnancy rates for high BMI IVF patients. ¹ Russo M, Ates S, Shaulox T, Dahan M H. Morbid obesity and pregnancy outcomes after single blastocyst transfer: a retrospective, North American study. J Assist Reprod Genet. 2017 April; 34(4):451-457. doi: 10.1007/s10815-017-0883-9. Epub 2017 Feb. 11. PMID: 28190215: PMCID: PMC5401700.

Minimization of blood presence during transfer: Use of steerability and visualization during the embryo transfer catheter placement minimizes trauma of the cervical canal, the outer and inner ostium and allows the physician to avoid contact with the endometrium present at time of transfer. This enhanced control minimizes contact that causes blood presence due to trauma occurring between the contact of the uterine wall and ET catheter, which can influence embryo attachment rates (Alvero et al., 2003; Tiras et al., 2012; Plowden et al., 2017; Singh et al., 2020).

Use of miniaturized components make visual embryo transfer possible: A small diameter is a critical specification in an ET catheter to insure avoidance of contractile response which decreases likelihood of pregnancy (Masroor M et al 2023)². Existing standard-of care ET catheters have <2.0 mm diameter, small enough to avoid contractile response, however they do not possess a camera for visualization or inflow and outflow ports to allow for distention and enhanced visualization. Miniaturization of cameras with the advent of sub 1 mm chip on tip camera technology and use of advanced materials such as Nitinol and new manufacturing methods to create micro channels in the catheter body allows for diameter catheter that is similar in size to existing ET catheters while also possessing camera and fluid delivery capability. ² Javedani Masroor M, Younesi Asl L, Sarchami N. The Effect of Uterine Contractions on Fertility Outcomes in Frozen Embryo Transfer Cycles: A Cohort Study. J Reprod Infertil. 2023 April-June; 24(2):132-138. doi: 10.18502/jri.v2412.12498. PMID: 37547572: PMCID: PMC10402453.

### Design and Construction

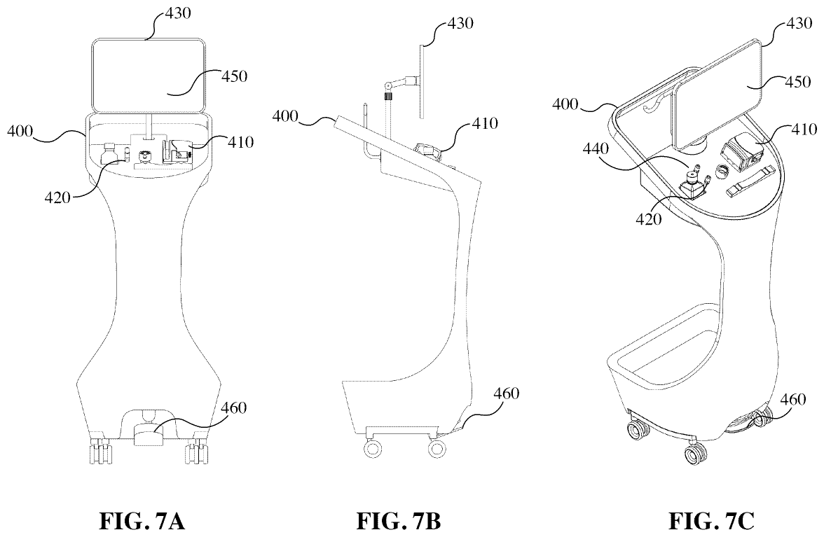

Embryo Transfer Management System (ETMS): Refer to FIG. 7A-7C. In another embodiment of the invention, the VET catheter described above is included as part of an Embryo Transfer Management System 400 which is an electromechanical system designed to enhance the precision and success of Visual Embryo Transfer procedures.

The ETMS 400 automates and regulates the fluid inflow and outflow, automates embryo dispensing, and incorporates a holder to stabilize the Handheld Assembly. Additionally it displays real time visualization on the touchscreen display 430 and includes a comprehensive user dashboard 450.

Precise Fluid Management: The ETMS offers precise fluid management capabilities, which are essential for maintaining optimal conditions within the uterus during the embryo transfer procedure. The system integrates a peristaltic pump 410, and a vacuum pump 420 to ensure smooth and steady fluid infusion and withdrawal. Pressure sensors monitor intrauterine pressure, maintaining a target range between 5 mmHg to 50 mmHg, while fluid scales accurately measure the fluid delivered to and collected from the uterus, calculating the fluid deficit. In one example fluid inflow rates are optimized at 0.6 mL/s and suction at 2 ml/s with a suction pressure of 100 mmHg. These settings allow the user to slowly inflate the uterine cavity to minimize the likelihood of causing discomfort to the patient during the procedure.

Visualization Display & Data Dashboard: The system provides a real-time feed, displaying live video from the camera 430 in the handheld assembly, allowing continuous monitoring of the procedure. The video feed may be done over Wi-Fi, Bluetooth or hard wired cable. The data dashboard shows real-time data on pressure, volume, and fluid balance. The intuitive touchscreen 450 interface offers different device modes, making it easy for users to operate the system.

Automated Embryo Dispensing: Automated embryo dispensing is a key feature of the ETMS. The system's automated mechanism ensures the accurate and controlled dispensing of the embryo into the uterus. The Automated Embryo Dispensing Function can be activated via a foot pedal or touchscreen control which creates a controlled release of the embryo into the uterus. The Automated Embryo Dispensing Function is performed by a programmable microfluidic pump 440 integrated into the ETMS. Specialized tubing links the microfluidic pump connector 440 on the ETMS to the proximal end of the embryo transfer catheter using a Luer lock 370 mechanism similar to those used in various medical tubing connections. In one example embodiment an infusion rate of 5-15 μl/s for 2-3 seconds These settings were established by measuring the pressure and outflow rate exerted in an embryo transfer catheter by multiple fertility doctors with high success rates. The measurements were conducted by connecting a pressure gauge inline with the embryo transfer catheter and the catheter syringe. The embryo transfer catheter was loaded with 20 μL of culture medium. Each doctor was then asked to push the plunger as they would during an actual embryo transfer. The pressure was recorded by the gauge, and the outflow rate was calculated by timing how long it took for the doctor to eject the 20 μL. These measurements were repeated at least 30 times for each doctor. The average outflow rate and pressure were found to be 10 μL/s, and 0.26 mmHg, respectively. No statistically significant differences were observed between doctors. Alternatively, the device can be calibrated to the dispensing settings of the particular physician if they prefer to modify the device for a customized setting or alternatively the physician may override the automated ET injection during the procedure.

Handheld Assembly Holder: A specialized holder is built into the top of the ETMS that secures the handheld visualization screen assembly at the top of the unit, reducing the need for manual handling and enhancing stability. This holder provides different degrees of mobility, including back and forth (longitudinal), lateral, angular (45 degrees up and down), and vertical adjustments. This multi-directional mobility allows the physician to position the handheld assembly precisely, ensuring optimal placement and stability during the procedure. Future versions of the ETMS may feature mechanized holders for automated movement and positioning, further enhancing precision and ease of use. The handheld Assembly Holder interfaces with the self-attachment strap which straps to the patient and accommodates patient movement during the procedure.

User Controls: User control of the ETMS is facilitated through a comprehensive touchscreen 450 interface, foot pedal control 460, and additional controls on the handheld portion of the VET System. This design allows for convenient and responsive operation during the procedure.

Mobile/Compact Unit: The ETMS is compact and mobile, with a total height not exceeding 48 inches, allowing it to be conveniently in proximity to the patient, eliminating the need for tubing or wires to extend across the procedure room. Equipped with wheels, the unit can be easily maneuvered within the clinical setting, enhancing its usability and flexibility.

In summary, in one embodiment, the ETMS consists of a peristaltic pump 410, vacuum pump 420, a syringe infusion pump 440, a handheld assembly holder at the top of the unit, in addition to pressure sensors and scales to measure the fluid delivered to the uterus and collected from the uterus to calculate fluid deficit, a touchscreen user interface and a microcontroller to manage the hardware and software. It is an optional component of the VET System used for improved control, delivery, and retrieval of fluid.

The control system can either be pre-programmed to provide fluid delivery and collection to assist with the embryo transfer procedure, or can be controlled by the operator.

In a pre-programmed procedure (i.e automated), the minimal volume of fluid will be delivered into the uterus for visualization. Approximately 1 cc to 10 cc of fluid will be needed for visualization, depending on uterine anatomy and cracking pressure of the fallopian tube. A target intrauterine pressure range of between 5 mmHg to 50 mmHg will be set in the fluid management system. Prior to the injection of the embryo into the uterus, the fluid management system will remove fluid from the uterus using active suction and verify all fluid has been collected. It is critical that fluid is removed from the uterus prior to transfer of the embryo to eliminate risk that the embryo becomes suspended in the fluid.

In a user operated procedure, the user will control fluid inflow and outflow using controls on the fluid management system display, a foot pedal control, or controls on the handheld portion of the VET System. In an alternate embodiment, user controls may be placed on the Handheld Assembly.

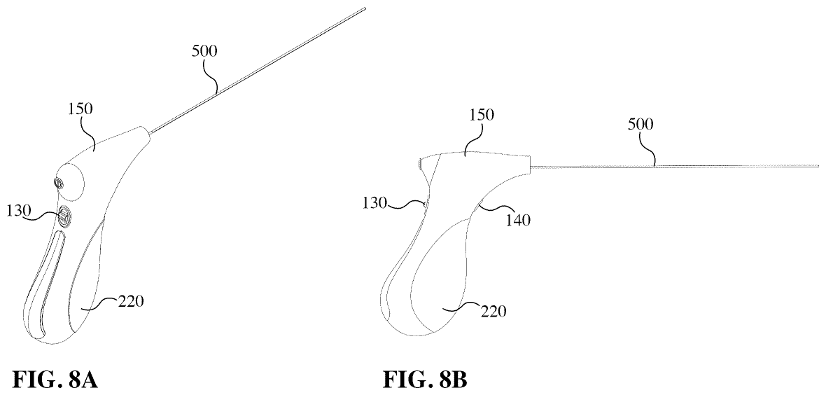

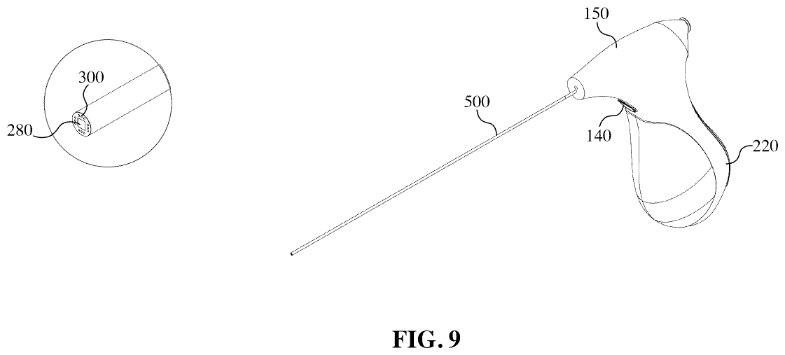

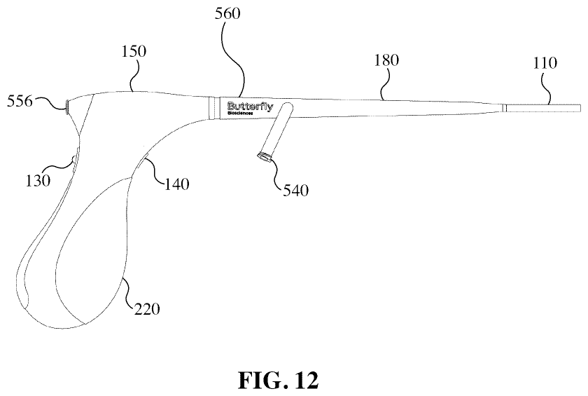

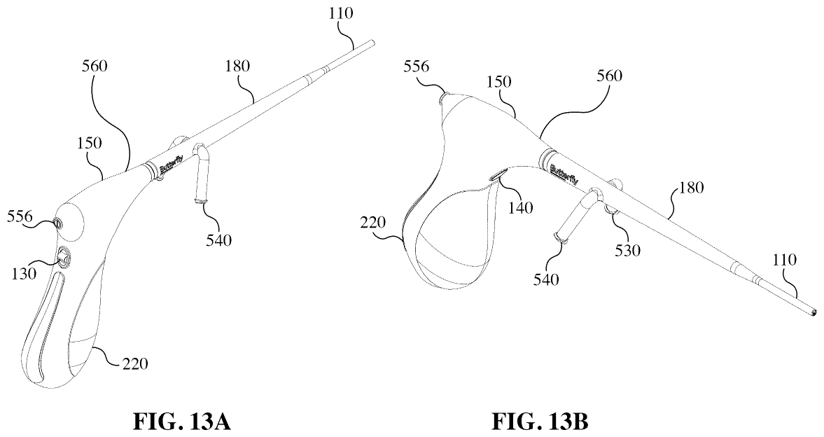

Handheld Assembly ETMS Version—Refer to FIGS. 8-13. Comprises the Reusable Handle 150 & Visualization Sheath 180, description provided in the following sections:

Reusable Handle 150—The reusable handle 150 of the Visual Embryo Transfer Handheld Assembly is an ergonomically designed component which serves as the primary interface between the physician and the advanced functionalities of the Visual Embryo Transfer system, providing a comfortable and intuitive control mechanism during the procedure.

The Reusable Handle contains a thin steerable shaft 500 of 1.17 mm diameter housing a high-resolution Complementary Metal-Oxide Semiconductor (CMOS; Omnivision, AMS or similar) based camera 280 with dimensions of <1 mm×1 mm×1 mm, and light illumination fibers. Illumination Fibers 300 are concentrically distributed around the CMOS camera 280 to evenly distribute light within the uterus. The illumination source for the Illumination Fibers 300 is located within the main body of the Reusable Handle.

Placing the LEDs into the reusable handle and providing illumination via fibers reduces the diameter of the overall device, and avoids exposing the uterus to the high direct heat produced by the LED. If the LEDs were to be placed at the distal end of the shaft, at least two LEDs would be required to get even light exposure across the uterus, and this increase the diameter of the shaft by 2-3 mm. A larger outer diameter increases discomfort to patients, thus increasing risk of error, complication, or failure of the embryo transfer procedure due to patient movement or uterine contractions. Additionally, because LEDs can generate heat sufficient to cause damage to patient tissue and embryos, use of Illumination Fibers 300 eliminates the risk from heat at the Visualization Sheath Tip 110 and improves the safety profile of the VET System.

The LED source located within the Reusable Handle 150 uses light in a range of wavelengths 550 nm. A higher wavelength, lower frequency LED is preferred for embryo safety. DNA damage and epigenetic issues can result from exposure of cells to UV light. The cut off for embryo DNA damage is approximately 500 nm. (Bodis et al, 2020; Bognar et al., 2019; Le et al, 2014, and Yamauchi et al, 2002, 2010) Use of LED light above 550 nm wavelength (e.g. orange or red light) improves the safety profile of the procedure. In an alternative configuration, standard LEDs may be utilized between 275-950 nm may be utilized.

Control over the movement of the thin shaft 500 is facilitated by the steerability control knob 130 located on the reusable handle. This knob allows the physician to make fine adjustments to the angle of the shaft, at an angle of up to +/−30 degrees or more.

The handle's outer casing 220 is molded from durable materials, providing a robust yet lightweight structure that is easy to handle during procedures. The ergonomic design of the outer molded handle ensures that it fits comfortably in the physician's hand, reducing fatigue.

To interface with the disposable outer sheath, the reusable handle has a mating adapter which allows for safe connection of the Reusable Handle and Outer Sheath. The connection mechanism is designed for easy attachment and detachment, allowing for quick preparation and clean-up.

Another vital component of the reusable handle is the embedded data processing unit with Wi-Fi connectivity. This unit processes the visual data captured by the CMOS camera and transmits it wirelessly to the visualization device. In an alternative embodiment, transmittal to a display may be done over a hard wired connection.

Power management within the handle is designed to be versatile, supporting both wired and battery-operated configurations. This flexibility ensures that the handle can be powered reliably in various clinical settings, whether through a direct connection to a power source or via rechargeable batteries.

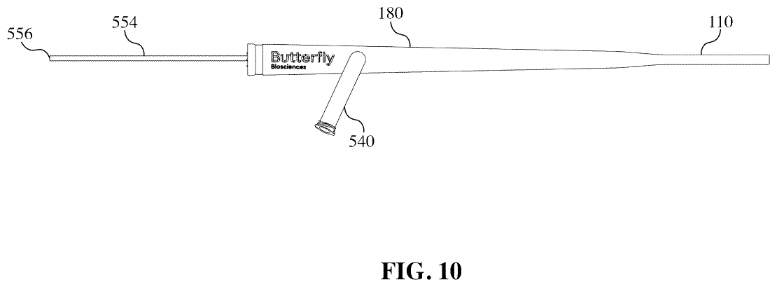

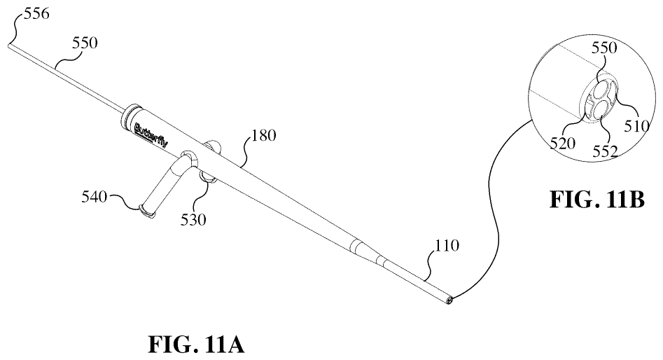

Disposable VET Outer Sheath-Refer to FIGS. 10-11—The disposable outer sheath 180 for the Visual Embryo Transfer Handheld Assembly is a single use sheath that contains the fluid and patient contacting components.

The outer sheath 180 slides over the steerable shaft 500 and locks into place against the reusable handle via the mating adapter. All components are made from biocompatible materials.

The sheath houses two fluid lumens 510 & 520 designed for the precise management of fluid inflow 510 and outflow 520. These lumens are engineered with smooth internal surfaces, free from corners or edges, to facilitate smooth and efficient fluid movement and reduce the risk of blockages. The inflow 510 and outflow lumens 520 are equipped with Luer lock connectors 530 and 540, ensuring a secure and leak-free connection to fluid sources. Additional lumens are reserved for the steerable shaft 552, and a dedicated lumen 550 & 554 for the embryo transfer catheter.

The outer sheath features a specialized tip 110 that can be controlled via the reusable handle. The tip of the outer sheath comprises the most distal 4 cm of the Sheath. The tip 110 is placed through the patient's cervix and positioned at the internal cervical ostium of the patient. The tip is 3-3.5 mm in diameter, is made from formable plastics (i.e PEBAX 4033, PEBAX 5533, PEBAX 6333, Tecoflex 100A or Tecoflex 60D, or similar) such that the steerable shaft of the Reusable Handle can drive articulation of the tip. The tip is approximately 2.5 mm to 4 mm and houses between two to four lumens. The most distal 1.5 cm to 3 cm of the Visualization Sheath 180 is adjustable by plus or minus 30 degrees relative to the central axis of the cannula to facilitate insertion of VET System into the patient. The dimensions are optimized for patient comfort as larger diameters can cause cramping or pain which may interfere with the success of the procedure.

This steerable tip 110 allows the physician to navigate the outer sheath through variable anatomy and properly position the tip of the embryo transfer catheter in the uterus. The tip's maneuverability ensures that the physician can reach the desired location for embryo transfer while avoiding anatomical obstacles.

Toward the proximal end of the Outer Sheath, there is a valve mechanism 556 for the introduction of the embryo transfer catheter. This valve 556 is designed to allow the catheter to be inserted smoothly into the uterine cavity while maintaining a sealed environment. The valve's construction is of a clear silicone that can prevent fluid backpressure but allow introduction of the embryo transfer catheter. In another embodiment, the valve mechanism may be a Tuohy Borst Seal, or a Hemostasis Valve.

To prevent re-use, the Outer Sheath has an embedded memory chip. When connected to the Reusable Handle, the reusable handle will register the serial number of the disposable and lock the user out should the Outer Sheath be re-used. This will ensure proper sterility.

Finally, the disposable outer sheath interfaces with the fluid management system through dedicated connectors and ports. These interfaces ensure seamless integration with the external fluid management components, allowing for precise control of fluid dynamics during the procedure. The sheath's design supports effective fluid distention of the uterine cavity, improving visualization and reducing the risk of complications such as fluid retention or expulsion of the embryo.

In summary, the disposable outer sheath for the Visual Embryo Transfer Handheld Assembly is a critical component that enhances the sterility, precision, and effectiveness of embryo transfer procedures. Its high-quality construction, secure connection to the reusable handle, advanced fluid management capabilities, and integrated tracking features make it an indispensable tool for physicians. By ensuring optimal conditions for visualization and embryo placement, the disposable outer sheath plays a pivotal role in improving patient outcomes in assisted reproductive technology.

Handheld Assembly, Non-ETMS Version: Comprises the Reusable Handle & Visualization Sheath, description provided in the following sections.

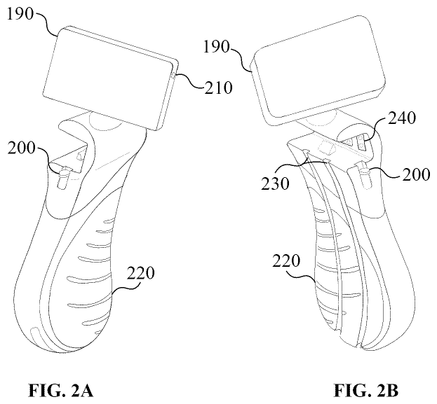

Reusable Handle: Refer to FIGS. 2A & 2B. The Reusable Handle 150 is the camera control unit of the embryo transfer system. The handle includes 1) a Video Display 190 mounted on a pivot for adjustable viewing angle, 2) internal hardware including image and video processing board, microcontrollers, rechargeable battery, local storage and wireless connectivity, 3) an outer enclosure with a handle containing an overmolded grip 220 for user comfort, and 4) a preloaded software interface.

The Video Display 190 is approximately 4 in by 3 in (101.6 mm by 76.2 mm) and has touchscreen controls, the overall length of the Reusable Handle 150 is approximately 13 in (330 mm). The handle is rechargeable via a USB-C port on the bottom of the handle.

The handle's main functions include camera controls (i.e. adjusting the light, focus, exposure, and other variable settings), providing electrical power to the hardware on the Visualization Sheath 180, processing inputs and outputs, data storage, wireless connectivity, and providing real time display of the procedure.

The handle connects with the Visualization Sheath 180 via 1) an electrical interface, and 2) a physical connection which holds the Visualization Sheath 180 firmly in place. Inputs and outputs and transferred across a pogo pin 240 interface at the site of connection between the Visualization Sheath 180 and Reusable Handle 150. Female pogo pins 240 are housed on the Reusable Handle 150. Additionally, the outer enclosure of the handle includes small grooves 230 on the side of the handle used to stow the tubing conducting fluid to and from the Visualization Sheath 180.

Visualization Sheath 180: The Visualization Sheath 180 is the outer casing and Embryo Transfer Catheter 100 guide of the VET System, and provides real time imaging, illumination, steerability, and multiple lumens to house the Scope Assembly 290, inflow/outflow, and to conduct the Embryo Transfer Catheter 100. The Visualization Sheath 180 has an entry point on the proximal end to allow for introduction of the soft Embryo Transfer Catheter 100 in either a preload or afterload technique, and prevents backflow of fluid around the Embryo Transfer Catheter 100.

The Visualization Sheath Tip 110 is the portion that is inserted into the patient. The Visualization Sheath Tip 110 is approximately 2.5 mm to 4 mm and houses between two to four lumens. The most distal 1.5 cm to 3 cm of the Visualization Sheath 180 is adjustable by plus or minus 30 degrees relative to the central axis of the cannula to facilitate insertion of VET System into the patient.

The Visualization Sheath 180 has between two to four lumens. The Scope Assembly Lumen 270 houses the Scope Assembly 290 comprising a CMOS Camera 280 and a set of Illumination Fibers 300 and electrical wire. A second lumen 340 is a dedicated lumen for the Embryo Transfer Catheter 100. A third lumen 330 is used for inflow and outflow of fluid. If a fourth lumen is used, it will be used to separate inflow and outflow. In another embodiment, the second lumen can house the Embryo Transfer Catheter 100, and be the source for fluid inflow and outflow, eliminating the need for additional lumens.

The dedicated lumen 340 for the Embryo Transfer Catheter 100 is optimal as it insulates the Embryo Transfer Catheter 100 from pressures or currents created by the inflow and outflow of fluid.

Fluid inflow can be provided via a 1 mL to 20 ml syringe, the VET Fluid Management System, or a programmed pump. Fluid collection can be performed using a 1 mL to 20 ml syringe, the VET Fluid Management System, a programmed suction pump, or gravity outflow via the fluid lumen 330 or cervical canal. The optimal suction pressure is 100 mmHg. In another embodiment, fluid inflow and outflow can be managed via button controls on the Reusable Handle 150 or Visualization Sheath 180 which activate inflow and outflow on command.

The camera used is a Complementary Metal-Oxide Semiconductor (CMOS; Omnivision, AMS or similar) based camera 280 with dimensions of less than 1 mm×1 mm×1 mm. Illumination Fibers 300 are concentrically distributed around the CMOS camera 280 to evenly distribute light within the uterus. The illumination source for the Illumination Fibers 300 is located within the main body of the Visualization Sheath 180. Use of fibers for illumination reduces the diameter necessary for the components, and avoids heat generated within the uterus by an LED light source placed at the Visualization Sheath Tip 110. Use of an LED would require at least two LEDs to provide appropriate illumination for the embryo transfer process, however this would increase the diameter of the Visualization Sheath 180 by at least 2 mm to 3 mm. A larger outer diameter increases discomfort to patients, thus increasing risk of error, complication, or failure of the embryo transfer procedure due to patient movement or uterine contractions. Additionally, because LEDs can generate heat sufficient to cause damage to patient tissue and embryos, use of Illumination Fibers 300 eliminates the risk from heat at the Visualization Sheath Tip 110 and improves the safety profile of the VET System.

The LED source located within the Visualization Sheath 180 uses light in a range of wavelengths 550 nm. A higher wavelength, lower frequency LED is preferred for embryo safety. DNA damage and epigenetic issues can result from exposure of cells to UV light. The cut off for embryo DNA damage is approximately 500 nm. (Bodis et al, 2020; Bognar et al., 2019; Le et al, 2014, and Yamauchi et al, 2002), 2010 Use of LED light above 550 nm wavelength (e.g. orange or red light) improves the safety profile of the procedure.

The body of the Visualization Sheath 180 is located at the proximal end and has two user controls: 1) Steering Lever 130 which articulates the Visualization Sheath Tip 110 by +/−30 degrees, and 2) the image and Image/Video Trigger 140. These user controls are located near the location that the user would grip the device to allow for device control with one hand.

Within the body of the Visualization Sheath 180, there is a manifold component that connects the multi-lumen distal tube to fluid inflow and outflow sources, and to the port through which the Embryo Transfer Catheter 100 is introduced.

The insertion point for the Embryo Transfer Catheter 100 is at a port on the proximal end of the Visualization Sheath 180 170. The port houses a silicone seal which is controlled by a screw cap or switch. When the screw cap is rotated, the Silicone seal either opens or closes depending on the pressure exerted on the silicone seal. The seal can be opened to introduce the soft Embryo Transfer Catheter 100 and then closed over the Stainless Steel Stiffening Rod 360 of the Embryo Transfer Catheter 100. This configuration provides mobility to the Embryo Transfer Catheter 100 within the device while preventing backflow of fluid during the procedure. In another embodiment, the silicone seal can be replaced with a balloon that can be inflated or deflated, thus providing a fluid tight seal.

Electrical power for the Visualization Sheath 180 is provided from the Reusable Handle 150. The Visualization Sheath 180 has a power and data exchange interface utilizing a standard electrical connector such as Pogo Pins 250, or a wired solution.

All materials used are biocompatible and mouse embryo assay tested. The Visualization Sheath 180 is either fully disposable, or can be reused.

During operation, the need to maintain the position of the tip placement becomes critical. Inadvertent movement of the Visualization Sheath Tip 110 increases risk that the embryo is transferred into the wrong location. To ensure that placement can be maintained, the device may utilize a cervical stopper or acorn seal that is placed up against the edge of the external cervical ostium. Alternatively, the Visualization Sheath 180 can be fitted with a deployable reverse umbrella seal which deploys at the internal cervical ostium to keep the sheath in place.

In another embodiment, the Visualization Sheath 180 may come with multiple LED sources, including LEDs that emit light with varying wavelengths not less than approximately 550 nm. In this embodiment, the user will control the color of the light source within the uterus.

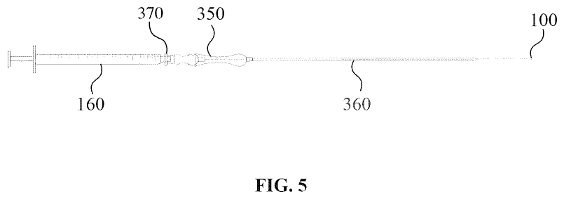

Embryo Transfer Catheter 100: Refer to FIG. 5. A specialized Embryo Transfer Catheter 100 has been designed to be compatible with the Visualization Sheath 180. The optimal Embryo Transfer Catheter 100 was designed with three primary considerations: 1) compatibility with Visualization Sheath 180, 2) ease of physician use and 3) ease of embryologist use.

The Embryo Transfer Catheter 100 has an outer diameter of approximately 0.91 mm, and inner diameter of approximately 0.56 mm and length of approximately 27 cm. The catheter has a soft surface made of polyurethane and may be extruded with air bubbles to provide echogenicity.

The Embryo Transfer Catheter 100 has a central lumen which extends along the length of the catheter. The tip of the inner cannula has a rounded, smooth, atraumatic edge. The catheter is reinforced with a Stainless-steel Stiffening Rod 360 that extends from the proximal end of the transfer catheter up to approximately 8 cm short of the catheter tip.

The embryo transfer also contains depth markers which are separated by 1 cm to help with positioning. Depth markers are provided on the proximal and distal ends of the transfer catheter. There are four distal markers, the first of which is visible the moment the inner cannula is 1 cm past the distal tip of the Visualization Sheath 180. Similarly, on the proximal end, when the first depth marker aligns with the proximal entry port on the Visualization Sheath 180, that will indicate that the tip of the inner cannula is flush with the Visualization Sheath 180 tip. The proximal depth markers will be visible on the metal stiffening rod.

A proximal hub 350 is provided to connect the soft catheter to the Embryo Transfer Catheter 100 Syringe 160. The ET Catheter Syringe 160 is used to provide the negative and positive pressure to load or expel the embryo. Embryos are loaded from the distal tip

When the Embryo Transfer Catheter 100 is packaged, it will be packaged with a Loading Sheath which is a polyethylene outer sheath that extends up to approximately 4 cm short of the Embryo Transfer Catheter 100 tip. The Loading Sheath facilitates loading of the embryo transfer by an embryologist by leaving a short segment of the Embryo Transfer Catheter 100 exposed. The loading sheath is removed prior to inserting the Embryo Transfer Catheter 100 into the Visualization Sheath 180.

### REFERENCES

For ease of reference, the following key identifies numerals on the figures and related items associated with those numerals:
Assembled Visual Embryo Transfer System—90
Embryo Transfer Catheter—100
Visualization Sheath Tip—110
Steerability Lever—130
Image/Video Trigger—140
Reusable Handle—150
Embryo Transfer Catheter Syringe—160
Proximal Seal of Visualization Sheath—170
Visualization Sheath—180
Video Display—190
Visualization Sheath Lock Switch—200
Reusable Handle On/Off Switch—210
Overmolded Plastic Grip—220
Grooves for Fluid Lines—230
Female Pogo Pins—240
Male Pogo Pin Connectors—250
Scope Assembly Lumen—270
CMOS Camera—280
Scope Assembly—290
Illumination Fiber Bundle—300
Distal Opening: ET Lumen—310
Inflow/Outflow Port—320
Inflow/Outflow Lumen—330
Dedicated ET Lumen—340
Proximal Hub of Embryo Transfer Catheter—350
Stainless Steel Stiffening Rod—360
Embryo Transfer Catheter Luer Lock—370
Embryo Transfer Management System—400
Peristaltic Pump—410
Vacuum Pump—420
Visualization Display—430
Embryo Dispensing Pump—440
Touchscreen Display—ETMS—450
ETMS Foot Pedal—460
Steerable Shaft—500
Dedicated Inflow Lumen—510
Dedicated Outflow Lumen—520
Inflow Luer Lock—530
Outflow Luer Lock—540
Embryo Transfer Catheter Lumen—Distal End 550
Steerable Shaft Lumen—552
Embryo Transfer Catheter Lumen—554
Embryo Transfer Catheter Lumen Valve—556
Handheld Assembly—ETMS Version—560

### Other Embodiments for VET System

The previous sections present the optimal configuration for a Visualized Embryo Transfer System. Other embodiments may be viable.

One embodiment does not make use of a Visualization Sheath 180. Rather the Embryo Transfer Catheter 100 is designed to be steerable, with a camera and light assembly placed at the distal end of the Embryo Transfer Catheter 100. This option ensures that the camera is located near the location of transfer whereas in the optimal configuration the distal end of the Visualization Sheath 180 is placed at the internal cervical ostium; further advancing the Visualization Sheath 180 past the internal os may cause bleeding or contraction. The Embryo Transfer Catheter 100 can have a double, triple lumen, or Scope Assembly 290. In the double lumen option, one lumen houses the embryo, and a second lumen houses the camera components and wire. In the triple lumen, one lumen houses the embryo, a second lumen houses the Scope Assembly 290, and the third lumen does inflow and outflow. In the quadruple lumen, one lumen houses the embryo, a second lumen houses the camera components and wire, and the third and fourth lumens are dedicated for inflow and outflow. The Embryo Transfer Catheter 100 can either be hardwired into an external display, or interface with the Reusable Handle 150. Using this device, the user will load the embryo into the Embryo Transfer Catheter 100, place the Embryo Transfer Catheter 100 into the uterus and expel the embryo.

In another embodiment, the embryo transfer system consists of a reusable steerable tube and distal tip assembly which houses a camera and illumination source (the “Scope Assembly 290”). A disposable tubular sleeve of two, three or four lumens will be placed over the reusable, steerable tube containing the Scope Assembly 290. In the double lumen construction, one lumen is used to house the reusable steerable tube and Scope Assembly 290, and the second lumen conducts the embryo or Embryo Transfer Catheter 100. In the triple lumen construction, one lumen houses the reusable steerable tube and Scope Assembly 290, the second lumen conducts the embryo or Embryo Transfer Catheter 100, and the third lumen conducts inflow and outflow of fluid. In the four lumen construction, one lumen houses the reusable steerable tube and Scope Assembly 290, the second lumen conducts the embryo or Embryo Transfer Catheter 100, and the third and fourth lumens conduct inflow and outflow of fluid.

In all possible configurations of the visualized embryo transfer, the location of the camera can also be moved from the distal tip to a fiber optic lens visualization system. For example, the tip of the scope can house an optical lens coupled with an optical fiber that carries the light to an eyepiece that can pair with a camera head that then carries the display to an external tower that performs image processing and digital display.

### Accessories

Mock Embryo Transfer Catheter: The mock embryo transfer catheter has the same specifications and description as the Embryo Transfer Catheter (previously described) but does not have a central lumen or distal opening for transporting the embryo.

Automated injector for consistent expulsion of embryo: To standardize the Embryo Transfer process, an automated injector can be placed on the Embryo Transfer Catheter 100 to control the injection pressure of the ET Catheter Syringe 160 on the Embryo Transfer Catheter 100. Significant operator variability results from varied injection pressures being applied at the time of embryo transfer. Applying too much pressure on the embryo transfer ET Catheter Syringe 160 will result in the embryo being transferred further away from the intended transfer location due to the momentum created by the injection pressure. This may cause an ectopic pregnancy. The automated injector fits over the 1 cc ET Catheter Syringe 160 and can be activated to inject the embryo at the time of transfer using controlled pressure.

Embryo Transfer Culture Medium—The Visual Embryo Transfer System is compatible with all embryo culture and embryo transfer medium. The medium of choice should contain water, bicarbonate or HEPES as buffer, salts, human serum albumin, hyaluronan, gentamicin, amino acids, an energy source and vitamins. Examples include G-2™, G-TL™, EmbryoGlue™, and UTM™ from Vitrolife, as well as ORIGIO Sequential Blast™ from CooperSurgical. Additionally, 500-700 IU of hCG, may be combined with 10-40 μl of medium to perform an intrauterine hCG injection 3 to 15 min before the embryo transfer.

VET optimized Culture Media: An alternative to commercially grade media with a custom formulation and packaging is also contemplated, that may include the following components at proprietary levels, and other additives:
Sodium bicarbonate
Sodium pyruvate
Gentamicin (50 μg/ml)
Water
Glucose
Sodium chloride
Calcium chloride
Potassium phosphate
Magnesium sulfate
Phenol red
Potassium chloride
Sodium lactate
Amino acids
Vitamins
Buffer (HEPES alone or in combination with Sodium Bicarbonate)
Hyaluronan (0.125-0.5 mg/ml)
Human Serum Albumin (HSA) (5 mg/ml)

Additives such as HSA and hCG may be included as part of a proprietary media. The media may be packaged in a unique cartridge design that is compatible only with the Embryo Transfer Management System.

Embryo Transfer Catheter Introducer: A separate introducer is provided to facilitate the passage of the Embryo Transfer Catheter tip through a seal, specifically a septum seal, during embryo transfer procedure. A common challenge of Visual Embryo Transfer is placing the thin flexible tip of the ET Catheter through the seal of the dedicated channel in the VET Outer Sheath. This is difficult due to the small diameter of the seal, and the lack of rigidity on the transfer catheter tip. The ET Catheter Introducer addresses this by providing an external introducer that covers the thin flexible tip of the ET catheter, allowing smooth and secure passage through the septum seal.

The Introducer comprises the Body, and the Pull-down tab at the most proximal end. The ET Catheter is loaded into the lumen of the body. The Body is a cylindrical tube with an internal diameter of 0.95-1.2 mm, slightly larger than the outer diameter on the ET Catheter. The length of the body is between 8-15 cm, sufficient to cover the exposed distal tip of the ET Catheter. Along the length of the Body is a slit which allows removal of the Introducer while the ET Catheter is in the dedicated lumen of the outer sheath.

With the introducer in place, the ET Catheter can be placed into the working channel seamlessly by the user. Once the introducer is past the seal, the ET Catheter can be pushed further into the lumen of VET Outer Sheath and the introducer removed via the Pull Tab.

Tubing Set: A separate tubing set is configured comprising biocompatible Tygon Tubing. The tubing set can contain tubing for inflow of fluid, outflow of fluid, and for connection to the fixture that regulates automated embryo dispensing from the Embryo Transfer Management System.

### Kit Contents

### Kit—Non ETMS Version

Embryo transfer catheter with loading sheath and Syringe
Visualization Sheath
1-20 cc Syringe
hCG wash for uterus
Embryo Transfer Culture Medium

### Kit—ETMS Version

Reusable Handle
Visual Embryo Transfer Outer Sheath
Embryo Transfer Catheter
Tubing Set
Proprietary embryo transfer culture medium in proprietary cartridge

### Functionality of the System

The following describes the key functions of the visualized embryo transfer system:
Real Time Visualization: The Visualization Sheath 180 (non ETMS version) and Reusable Handle 150 (ETMS version) incorporate a CMOS camera 280 and illumination to permit real-time visualization of the uterine environment during the embryo transfer. This allows the clinician to place the embryo in the optimal location within the uterus for transfer. The visualization works when the Visualization Sheath 180 is connected to the Reusable Handle 150. The real-time view is displayed on the Video Display 190 on the Reusable Handle 150 (non ETMS Version) or on 430 (ETMS Version), and can also be displayed on an external monitor. Additionally, because the Embryo Transfer Catheter 100 has echogenicity built in, ultrasound visualization can be used to complement real-time visualization.
Embryo Handling and Delivery: The system includes an echogenic Embryo Transfer Catheter 100 that is designed for ease of loading and compatibility with the Visualization Sheath 180. This Embryo Transfer Catheter 100 enters into a dedicated lumen 340 (non-ETMS version) or 550 (ETMS version) within the Visualization Sheath 180 and can traverse the lumen to reach the distal end of the Visualization Sheath Tip 110, and past the Visualization Sheath Tip 110 into the uterus.

The construction of the Embryo Transfer Catheter 100 facilitates embryo loading, and placement of the Embryo Transfer Catheter 100 into the Visualization Sheath 180. The Embryo Transfer Catheter 100 has a central single lumen to pick up the embryo from the petri dish and deposit the embryo into the uterus. The pressure to pick up and deposit the embryo is provided from a 1 cc ET Catheter Syringe 160 that is attached to the proximal hub 350 placed on the proximal end of the inner cannula of the Visualization Sheath 180. The Embryo Transfer Catheter 100 comes packaged with a removable embryo loading sheath which provides rigidity up to four centimeters short of the embryo transfer tip to the portion section of the inner lumen that is not reinforced by the stainless steel stiffening rod 360. With the loading sheath only the most distal 4 cm of the inner cannula is exposed. This distal portion of the Embryo Transfer Catheter 100 is then placed into the culture drop housing the embryo for pick up.

Using the embryo loading sheath the user can perform an ultrasound guided embryo transfer as the embryo loading sheath also serves as an outer sheath.

If the user wants direct imaging of the embryo transfer, the embryo loading sheath will be removed and the Embryo Transfer Catheter 100 will be placed into the Visualization Sheath 180 from the proximal end 170 (non-ETMS version) or 556 (ETMS version). The proximal end 170 of the Visualization Sheath 180 is fitted with a seal that can either open or close with a switch or knob (e.g. a Tuohy Bore Seal) in the non-ETMS version of the visual embryo transfer system. Alternatively, the proximal end 556 of the ETMS version of the Visualization Sheath 180 is fitted with a silicone seal with a slit from which the embryo transfer catheter can be introduced. A user-controlled seal is essential as the distal tips of embryo transfer catheters have insufficient rigidity to pierce a hard seal. The seal is opened to introduce the Embryo Transfer Catheter 100 and once the stainless-steel stiffening rod 360 (located on the Embryo Transfer Catheter 100) is aligned with the silicone seal, the seal can be closed to ensure a fluid and air tight lumen. This construction allows the physician to perform a mock embryo transfer, pre-load, or afterload embryo transfer.
Fluid Management Controls: The Visualization Sheath 180 has fluid inflow and outflow lumens 330 (non-ETMS version), 510 and 520 (ETMS version) to introduce and remove fluid from the uterus. In some embodiments, the inflow and outflow lumen is the same lumen used to introduce the Embryo Transfer Catheter 100, and in other embodiments the inflow and outflow lumens are dedicated fluid lumens 510 and 520 such as in the ETMS version. Fluid is needed inside of the uterus to expand the endometrium thus providing illumination. However, fluid should not be present in the uterus when the embryo is transferred as the embryo may become suspended in the fluid and move to an unintended location. A minimal volume of fluid in the range of 1 cc to 10 cc is needed to allow visualization and must be fully removed from the uterus to perform a reliable embryo transfer procedure.
Steerability: The Visualization Sheath Tip 110 is flexible and can articulate plus or minus 30 degrees from the central axis of the cannula. This “steerability” function is user controlled, and retains the deflection angle even if the user's finger is removed from the Steering Lever 130. In the non-ETMS version, steerability is built into the Visualization Sheath Tip 110, whereas in the ETMS version steerability is provided by the steerable shaft 500.
User Interface: The VET system contains a Video Display 190 or 430 which allows the clinician to control the embedded imaging system and view or record the transfer procedure. The user interface displays real-time procedure data, allows touchscreen control of light intensity, autofocus adjustments, and automatic light exposure. Additionally, the user can perform camera calibrations from the touchscreen to ensure image fidelity.
Data Analysis and Guidance: An advanced system may include features that can analyze the embryo, or the imaging data and provide guidance to the clinician. For example, the System may have an internal or cloud-based algorithm that can assess the viability of the embryo prior to transfer based on morphological or morphokinetic characteristics. Doing so would allow the embryologist to assess the likelihood of embryo implantation prior to transfer. Alternatively, the System could also have an algorithm that assesses the endometrial imaging data from the Visualization Sheath 180 and can advise the user which uterine segment to transfer the embryo for the best results. As the system is a cloud-connected device, all data gathered from the procedure is collected and housed in the cloud.
Data Recording: The device may also record image and video data from the embryo transfer. These images and videos are stored on the local device and transferred to the Cloud System (SaaS) which houses a master archive of patient data for the user. Additionally, the device records basic information such as date/time, geographic location, disposable Visualization Sheath 180 UID, and procedure times.
Integration with Other Systems: The VET system is designed to integrate with other systems. This includes the clinic electronic medical records system which can receive the data recorded into the system thus eliminating the manual workflow associated with this process. Additionally, the System could be integrated with cloud-based platforms that perform embryo assessments, endometrial assessment, or endometrial receptivity assessment.
Wireless Connectivity: The Reusable Handle 150 has wireless connectivity and is used to create a secure HIPAA (Health Information Portability and Accountability Act) compliant connection to an online SaaS (software-as-a-service) application. The application captures patient records that detail the VET procedure. These data are placed into the physician/provider portal and become available for the physician office to access. Physician offices will utilize access to the patient record to review the procedure details, review and discuss the procedure with the patient and make notations either in a voice-dictated or written format. The record of each VET procedure is also de-identified and placed into a central repository which is accessible to Butterfly Biosciences. Prior to capture of a patient record in the de-identified database, consents for such access are acquired from the patient and the provider. The Company will utilize the consolidated repository of VET procedures to gain visibility into the VET procedure effectiveness. The Company will also utilize the repository of data, which includes visual images of uterine anatomies, to characterize uterine anatomies. This work has not been possible to date as no capability to consolidate repositories of this type exists today.

### Method of Use

Novel methods of use have been developed for use with the VET System. The VET System is compatible with preload or afterload transfer techniques.

Prior art devices are single lumen instruments which deposit an embryo into the uterus with a microdroplet of fluid. A challenge of performing visualized embryo transfer is in obtaining visualization of the uterine cavity with fluid and removing said fluid from the uterus prior to transferring the embryo while keeping the Embryo Transfer Catheter 100 in place at the optimal location within the uterus.

### Day of Procedure

### Optional Pre-Step 1: hCG Wash of the Endometrium

Approximately 3 to 15 minutes prior to the embryo transfer, an optional hCG wash may be performed using 40 μl of the embryo transfer medium supplemented with 500-700 IU of hCG (as previously described). The uterus can be flushed with the Visualization Sheath 180.

Alternatively, the embryologist may mix the hCG (i.e Pregnyl by Organon; Ovitrelle by Merck; Choriomon by IBSA or other manufacturers) into another culture medium or saline solution. The most effective dose of hCG for intrauterine infusion/injection is ≥500 IU contained in 10-40 μl of embryo culture media or saline solution (Conforti et al., 2022; Craciunas et al., 2018).

### Optional Pre-Step 2: Plasma Rich Platelet (PRP) Wash of the Uterus

Approximately 15 to 30 minutes prior to the embryo transfer, an optional PRP wash can be done of the uterus. PRP will be prepared from the patient's serum and placed into the uterus. This PRP wash can be done simultaneously with the hCG wash of the endometrium.

Optional Pre-Step 3: Wash of the Uterus with Proprietary Substances

Pre-wash of the uterus has been described with hCG and PRP, however other formulations can be utilized for the optional pre-wash step to improve likelihood of a successful outcome.

### Airless Embryo Transfer Load:

An airless ET catheter configuration is required for the Visual Embryo Transfer system to obviate movement of embryos within the ET catheter due to pressures that may exist in the uterus.

By employing this airless method to prepare the ET catheter we ensure the stability of the embryo throughout the VET process, even when injecting fluid volumes well above the typical requirement of 1-10 ml. In this sense, before loading the embryo into the catheter, all air is purged from the catheter by aspirating with the syringe a volume of medium that is slightly larger than the volume of the catheter employed. This is followed by connecting the catheter and fully depressing the plunger. This process involves adding two straightforward steps to the conventional catheter loading method, as illustrated in the accompanying figure. This method also guarantees that the volume of medium injected from the catheter into the uterus will be only that which contains the embryo and no more than that.

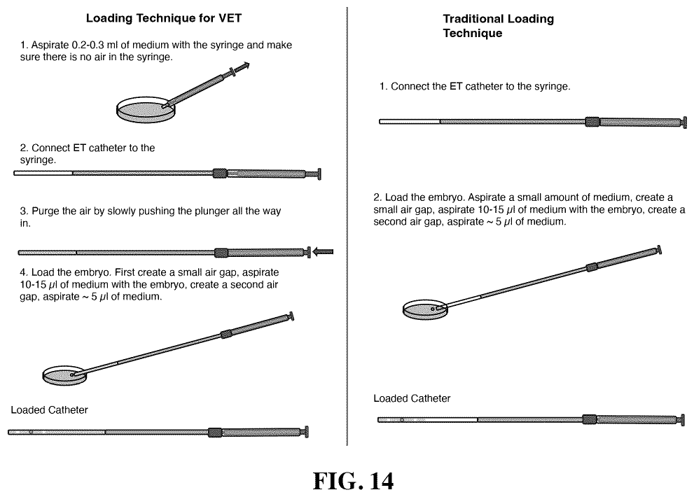

FIG. 14 illustrates a comparison of a catheter loading method for the VET system (left side) in accordance with various embodiments and a traditional catheter loading method (right side). In various embodiments, the embodiment method may reduce the air inside of the catheter which may help to keep the embryo stable during the VET procedure.

### Embryo Transfer:

The VET system is compatible with preload and afterload embryo transfer techniques. The following describes how each protocol is performed.

### Preload Technique:

Disinfect the Reusable Handle 150, and place in sterile field.

Enter unique patient identifiers that may include patient first name, last name, date of birth or patient ID number assigned by the physician office into the Reusable Handle 150 or the Touchscreen Display 450 of the ETMS.

Open the sterile one-time-use Visualization Sheath 180 and attach it to the Reusable Handle 150.

Power on the assembled VET System 90 or 560 and verify the function of the camera, steering, and illumination

Connect the Visualization Sheath 180 to inflow and outflow sources. If the VET Fluid Management System is used, connect the Visualization Sheath 180 Tubing to the VET Fluid Management System.

Thaw the embryo(s) to be transferred (lab step).

Load the embryo(s) to be transferred into the Embryo Transfer Catheter 100 (lab step).

Remove the Loading Sheath from the Embryo Transfer Catheter 100.

Load Embryo Transfer Catheter 100 into Visualization Sheath 180. In the non-ETMS version, open the proximal seal to allow entry of the ET Catheter. In the ETMS version, utilize the introducer to allow entry of the ET catheter through the Silicone Seal 556. Advance the ET Catheter into the Visualization Sheath 180 until the Stainless Steel Stiffening Rod 360 of the ET Catheter is in contact with the seal. Ensure there is a proper seal prior to advancing the ET Catheter further.

Place the Assembled VET System 90 or 560 into the uterus. Use the Steering Lever 130 if anatomy is atypical.

Insufflate the uterus with approximately 1 cc to 10 cc of fluid.

Advance and extend the Embryo Transfer Catheter 100 into the uterus and position the tip of the catheter over the optimal location for embryo transfer.

Remove all fluid from the uterus and ensure that all fluid is removed from the uterus using the Embryo Transfer Management System (ETMS). Or use abdominal ultrasound to confirm that all fluid has been removed from the uterus.

Deposit the embryo by activating the automated injector function on the ETMS. In an alternative embodiment (if ET done manually), press the injector on the ET catheter syringe 160 to insert embryo into the uterus.

Remove the VET System from the uterus, and confirm that the embryo is no longer present in the ET Catheter.

### Afterload Technique (Version 1—Fluid Removed Prior to Embryo Dispensing)

Disinfect the Reusable Handle 150 and place it in the sterile field.

Enter unique patient identifiers that may include patient first name, last name, date of birth or patient ID number assigned by the physician office into the Reusable Handle 150 or the Touchscreen Display 450 of the ETMS.

Open the sterile one-time-use Visualization Sheath 180 and attach it to the Reusable Handle 150.

Turn on the Handheld Assembly 90 or 560 and verify the function of the camera, steering, and light sources.

Connect the Visualization Sheath 180 to inflow and outflow sources. If the VET Fluid Management System is used, connect the Visualization Sheath 180 Tubing to the VET Fluid Management System.

Thaw the embryo(s) to be transferred (lab step) (lab step).

Place the mock Embryo Transfer Catheter into Visualization Sheath 180, using the proximal seal 170 or 556 as the entry point. Ensure a tight seal is maintained once the stainless steel stiffening rod 360 of the mock transfer catheter is in line with the seal. Alternatively a mock embryo transfer catheter may not be used.

Place the Handheld Assembly 90 or 560 into the uterus with mock transfer catheter. Use the Steering Lever 130 if anatomy is atypical.

Insufflate the uterus with 1 cc to 10 cc of fluid.

Identify target location for transfer. If a mock transfer is being performed, advance and extend the mock Embryo Transfer Catheter 100 into the uterus over the optimal transfer location. Take note of the distance markers on the mock device.

Remove the mock Embryo Transfer Catheter 100 from the Visualization Sheath 180.

Load the thawed embryo into the Embryo Transfer Catheter 100 (lab step).

Place the tip of the Embryo Transfer Catheter over the optimal location for embryo transfer, utilize distance markers to ensure precise placement.

Remove all fluid from the uterus and ensure that all fluid is removed from the uterus using the Embryo Transfer Management System (ETMS). Or use abdominal ultrasound to confirm that all fluid has been removed from the uterus.

Deposit the embryo by activating the automated injector function on the ETMS. In an alternative embodiment (if ET done manually), press the injector on the ET Catheter Syringe 160 to insert the embryo into the uterus.

Remove the VET Handheld Assembly 90 or 560 from the uterus, confirm that the embryo is no longer present in the ET Catheter.

### Afterload Technique (Version 2—Fluid Removed Prior to Placement of Live ET Catheter)

Disinfect the Reusable Handle 150 and place it in the sterile field.

Enter unique patient identifiers that may include patient first name, last name, date of birth or patient ID number assigned by the physician office into the Reusable Handle 150 or the Touchscreen Display 450 of the ETMS.

Open the sterile one-time-use Visualization Sheath 180 and attach it to the Reusable Handle 150.

Turn on the VET Handheld Assembly 90 or 560 and verify the function of the camera, steering, and light sources.

Connect the Visualization Sheath 180 to inflow and outflow sources. If the VET Fluid Management System is used, connect the Visualization Sheath 180 Tubing to the VET Fluid Management System.

Thaw the embryo(s) to be transferred (lab step) (lab step).

Place the mock Embryo Transfer Catheter into Visualization Sheath 180, using the proximal seal 170 or 556 as the entry point. Ensure a tight seal is maintained once the stainless steel stiffening rod 360 of the mock transfer catheter is in line with the seal. Alternatively a mock embryo transfer catheter may not be used.

Place the Handheld Assembly 90 or 560 into the uterus with mock transfer catheter. Use the Steering Lever 130 if anatomy is atypical.

Insufflate the uterus with 1 cc to 10 cc of fluid.

Identify target location for transfer. If a mock transfer is being performed, advance and extend the mock Embryo Transfer Catheter 100 into the uterus over the optimal transfer location. Take note of the distance markers on the mock device.

Remove the mock Embryo Transfer Catheter 100 from the Visualization Sheath 180.

Remove all fluid from the uterus and ensure that all fluid is removed from the uterus using the Embryo Transfer Management System (ETMS). Or use abdominal ultrasound to confirm that all fluid has been removed from the uterus.

Load the thawed embryo into the Embryo Transfer Catheter 100 (lab step). Use the distance markers on the ET Catheter for reference in placement of the ET Catheter tip.

Deposit the embryo by activating the automated injector function on the ETMS. In an alternative embodiment (if ET done manually), press the injector on the ET Catheter Syringe 160 to insert the embryo into the uterus.

Remove the VET Handheld Assembly from the uterus, confirm that the embryo is no longer present in the ET Catheter.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. Various embodiments of the present disclosure include variations on described embodiments that would be apparent to the skilled addressee, including variations obtained by: replacing features, elements and/or acts with equivalent features, elements and/or acts; mixing and matching of features, elements and/or acts from different described embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

### BIBLIOGRAPHY

Alvero, R., R. M. Hearns-Stokes, W. H. Catherino, M. P. Leondires, and J. H. Segars. 2003. “The Presence of Blood in the Transfer Catheter Negatively Influences Outcome at Embryo Transfer*.” Human Reproduction 18 (9): 1848-52. https://doi.org/10.1093/humrep/deg359.
Arora, Puneet, and Vandana Mishra. 2018. “Difficult Embryo Transfer: A Systematic Review.” Journal of Human Reproductive Sciences 11 (3): 229-35. https://doi.org/10.4103/jhrs.JHRS_59_18.
Berkkanoglu, M., M. Isikoglu, M. Seleker, and K. Ozgur. 2006. “Flushing the Endometrium Prior to the Embryo Transfer Does Not Affect the Pregnancy Rate.” Reproductive Biomedicine Online 13 (2): 268-71. https://doi.org/10.1016/s1472-6483 (10) 60625-6.
Cirillo, F, P Patrizio, M Baccini, E Morenghi, C Ronchetti, L Cafaro, E Zannoni, A Baggiani, and P E Levi-Setti. 2020. “The Human Factor: Does the Operator Performing the Embryo Transfer Significantly Impact the Cycle Outcome?” Human Reproduction 35 (2): 275-82. https://doi.org/10.1093/humrep/dez290.
Cirillo, Federico, Pasquale Patrizio, Emanuela Morenghi, Michela Baccini, Elena Zannoni, Luca Cafaro, Camilla Ronchetti, Annamaria Baggiani, and Paolo Emanuele Levi Setti. 2019. “Does the Operator Performing the Embryo Transfer Significantly Influence the Cycle Outcome?” Fertility and Sterility 112 (3): e69-70. https://doi.org/10.1016/j.fertnstert.2019.07.303.
Cirillo, Federico, Daria Spadaro, Emanuela Morenghi, Michela Baccini, Andrea Busnelli, Camilla Ronchetti, Elena Albani, Valentina Parini, Pasquale Patrizio, and Paolo Emanuele Levi-Setti. 2022. “Different Actors for the Same Play: The Impact of the Embryologist Performing the Embryo Transfer.” Reproductive Biomedicine Online 45 (4): 661-68. https://doi.org/10.1016/j.rbmo.2022.06.002.
Coroleu, Buenaventura, Pedro N. Barri, Olga Carreras, Francisca Martínez, Anna Veiga, and Juan Balasch. 2002. “The Usefulness of Ultrasound Guidance in Frozen-Thawed Embryo Transfer: A Prospective Randomized Clinical Trial.” Human Reproduction (Oxford, England) 17 (11): 2885-90. https://doi.org/10.1093/humrep/17.11.2885.
Chung, Cathy Hoi Sze, Alice Wai Yee Wong, Carol Pui Shan Chan, Sotirios H. Saravelos, Grace Wing Shan Kong, Lai Ping Cheung, Jacqueline Pui Wah Chung, and Tin Chiu Li. 2017. “The Changing Pattern of Uterine Contractions before and after Fresh Embryo Transfer and Its Relation to Clinical Outcome.” Reproductive Biomedicine Online 34 (3): 240-47. https://doi.org/10.1016/j.rbmo.2016.12.011.
Conforti, Alessandro, Salvatore Longobardi, Luigi Carbone, Giuseppe Gabriele Iorio, Federica Cariati, Maria Rosaria Campitiello, Ida Strina, Michela Palese, Thomas D'Hooghe, and Carlo Alviggi. 2022. “Does Intrauterine Injection of hCG Improve IVF Outcome? A Systematic Review and a Meta-Analysis.” International Journal of Molecular Sciences 23 (20): 12193. https://doi.org/10.3390/ijms232012193.
Craciunas, Laurentiu, Nikolaos Tsampras, Nick Raine-Fenning, and Arri Coomarasamy. 2018. “Intrauterine Administration of Human Chorionic Gonadotropin (hCG) for Subfertile Women Undergoing Assisted Reproduction.” The Cochrane Database of Systematic Reviews 10 (10): CD011537. https://doi.org/10.1002/14651858.CD011537.pub3.
Fanchin, R., C. Righini, F. Olivennes, S. Taylor, D. de Ziegler, and R. Frydman. 1998. “Uterine Contractions at the Time of Embryo Transfer Alter Pregnancy Rates after In-Vitro Fertilization.” Human Reproduction (Oxford, England) 13 (7): 1968-74. https://doi.org/10.1093/humrep/13.7.1968.
Gaast, M. H. van der, K. Beier-Hellwig, B. C. J. M. Fauser, H. M. Beier, and N. S. Macklon. 2003. “Endometrial Secretion Aspiration Prior to Embryo Transfer Does Not Reduce Implantation Rates.” Reproductive Biomedicine Online 7 (1): 105-9. https://doi.org/10.1016/s1472-6483 (10) 61737-3.
Ghaffari, Firouzeh, Kiandokht Kiani, Akram Bahmanabadi, and Mohammadreza Akhoond. 2013. “Comparison of Easy and Difficult Embryo Transfer Outcomes in in Vitro Fertilization Cycles.” International Journal of Fertility & Sterility 6 (4): 232-37.
Hou, Zhaojuan, Aihua He, Qiong Zhang, Nenghui Liu, Donge Liu, Yumei Li, Bin Xu, et al. 2022. “Endometrial Fluid Aspiration Immediately Prior to Embryo Transfer Does Not Affect IVF/Vitrified-Warmed Embryo Transfer Outcomes—a Prospective Matched Cohort Study.” Reproductive Biomedicine Online 44 (3): 486-93. https://doi.org/10.1016/j.rbmo.2021.11.004.
Javedani Masroor, Mojgan, Ladan Younesi Asl, and Niloufar Sarchami. 2023. “The Effect of Uterine Contractions on Fertility Outcomes in Frozen Embryo Transfer Cycles: A Cohort Study.” Journal of Reproduction & Infertility 24 (2): 132-38. https://doi.org/10.18502/jri.v24i2.12498.
Kava-Braverman, Alejandro, Francisca Martínez, Ignacio Rodriguez, Manuel Álvarez, Pedro N. Barri, and Buenaventura Coroleu. 2017. “What Is a Difficult Transfer? Analysis of 7,714 Embryo Transfers: The Impact of Maneuvers during Embryo Transfers on Pregnancy Rate and a Proposal of Objective Assessment.” Fertility and Sterility 107 (3): 657-663.e1. https://doi.org/10.1016/j.fertnstert.2016.11.020.
Larue, Lionel, Laure Bernard, Julie Moulin, Anne Massari, Nino-Guy Cassuto, Dominique Bouret, and Gwenola Keromnes. 2021. “Evaluation of a Strategy for Difficult Embryo Transfers from a Prospective Series of 2,046 Transfers.” F&S Reports 2 (1): 43-49. https://doi.org/10.1016/j.xfre.2020.11.004.
Listijono, Dave R., Tim Boylan, Simon Cooke, Suha Kilani, and Michael G. Chapman. 2013. “An Analysis of the Impact of Embryo Transfer Difficulty on Live Birth Rates, Using a Standardised Grading System.” Human Fertility (Cambridge, England) 16 (3): 211-14. https://doi.org/10.3109/14647273.2013.804956.
Mains, Lindsay, and Bradley J. Van Voorhis. 2010. “Optimizing the Technique of Embryo Transfer.” Fertility and Sterility 94 (3): 785-90. https://doi.org/10.1016/j.fertnstert.2010.03.030.
Mizrachi, Yossi, and Dana B. McQueen. 2022. “Embryo Transfer Success: It Is in Our Hands.” Fertility and Sterility 118 (5): 815-19. https://doi.org/10.1016/j.fertnstert.2022.08.858.
Plowden, Torie C., Micah J. Hill, Shana M. Miles, Benjamin Hoyt, Belinda Yauger, James H. Segars, John M. Csokmay, and Rebecca J. Chason. 2017. “Does the Presence of Blood in the Catheter or theDegree of Difficulty of Embryo Transfer Affect Live Birth?” Reproductive Sciences 24 (5): 726-30. https://doi.org/10.1177/1933719116667607.
Russo, Miguel, Senem Ates, Talya Shaulov, and Michael H. Dahan. 2017. “Morbid Obesity and Pregnancy Outcomes after Single Blastocyst Transfer: A Retrospective, North American Study.” Journal of Assisted Reproduction and Genetics 34 (4): 451-57. https://doi.org/10.1007/s10815-017-0883-9.
Saraee, Farnoosh, Faezeh Shekari, Ashraf Moini, Marya Sadeghi, Pooneh Ghaznavi, Abdoreza Nazari, Azadeh Ghaheri, Mehdi Totonchi, and Poopak Eftekhari-Yazdi. 2022. “Human Uterine Fluid Lavage-Derived Extracellular Vesicle Isolation: A Comparative Study for Minimally Invasive Endometrial Receptivity Assessment.” Reproductive Biomedicine Online 45 (3): 457-72. https://doi.org/10.1016/j.rbmo.2022.05.005.
Singh, Nidhi, Gita Khanna, Rohit Rao Pushkar, Rajshri Bagchi, Beetu Lamba, Archana Shakya, and Nija Rajbhandari. 2020. “To Study the Impact of Blood and Mucus on Embryo Transfer Catheter Tip and IVF Outcome.” Fertility Science and Research 7 (2): 204. https://doi.org/10.4103/fsr.fsr_43_20.
Tiras, Bulent, Umit Korucuoglu, Mehtap Polat, Ayse Saltik, Hulusi Bulent Zeyneloglu, and Hakan Yarali. 2012. “Effect of Blood and Mucus on the Success Rates of Embryo Transfers.” European Journal of Obstetrics & Gynecology and Reproductive Biology 165 (2): 239-42. https://doi.org/10.1016/j.ejogrb.2012.07.032.
Tomás, Candido, Kimmo Tikkinen, Leena Tuomivaara, Juha S. Tapanainen, and Hannu Martikainen. 2002. “TheDegree of Difficulty of Embryo Transfer Is an Independent Factor for Predicting Pregnancy.” Human Reproduction (Oxford, England) 17 (10): 2632-35. https://doi.org/10.1093/humrep/17.10.2632.
Zhu, L., L. Xiao, H. S. Che, Y. P. Li, and J. T. Liao. 2014. “Uterine Peristalsis Exerts Control over Fluid Migration after Mock Embryo Transfer.” Human Reproduction 29 (2): 279-85. https://doi.org/10.1093/humrep/det429.

1. A visual embryo transfer catheter system comprising:
a handle including a video display for displaying the interior of a uterus of a patient upon imaging of the uterus with a camera;
a scope assembly comprising a camera and an illumination source;
an embryo transfer catheter which is configured to deliver an embryo to the uterus upon imaging of the uterus with the camera when the uterus is illuminated by the light source; and
a visualization sheath which is configured to be coupled to the handle and to be inserted into the uterus of a patient, the sheath comprising at least three lumens, a first lumen for the scope assembly, a second lumen to permit passage of the embryo transfer catheter therethrough, and a third fluid supply lumen for inflow and outflow of fluid to and from the uterus.

2. The visual embryo transfer catheter system of claim 1, wherein the camera comprises a CMOS camera.

3. The visual embryo transfer catheter of claim 2, wherein the illumination source comprises illumination fibers that are concentrically distributed around the CMOS camera to evenly distribute light within the uterus.

4. The visual embryo transfer catheter of claim 3, wherein the illumination source comprises a range of wavelengths greater than approximately 550 nm to avoid damage to an embryo placed into the uterus.

5. The visual embryo transfer catheter system of claim 1, wherein the handle includes internal hardware including an image processing board, at least one microcontroller, a rechargeable battery, and a wireless connectivity unit.

6. The visual embryo transfer catheter system of claim 1, wherein the handle is configured to reusably couple to the visualization sheath.

7. The visual embryo transfer catheter system of claim 1, wherein the visualization sheath has a lever which is configured to articulate the distal tip of the visualization sheath by plus or minus 30 degrees from the radial axis of the main cannula.

8. The visual embryo transfer catheter system of claim 1, wherein the second lumen of the visualization sheath comprises a port that houses a silicone seal which is controlled by a screw cap such that when the screw cap is rotated, the silicone seal either opens or closes depending on the pressure exerted from the screw cap.

9. The visual embryo transfer catheter of claim 1, wherein the wireless connectivity unit of the handle is used to create a secure HIPAA compliant connection to an online software-as-a-service application.

10. The visual embryo transfer catheter system of claim 9, wherein the SaaS application captures patient data that detail a visual embryo transfer procedure.

11. The visual embryo transfer catheter system of claim 10, further comprising a physician portal for storing the patient data to allow a physician office to access the patient data.

12. A method for visual embryo transfer into a uterus of a patient comprising:
using a handle including a video display for displaying the interior of a uterus of a patient upon imaging of the uterus with a camera and a visualization sheath which is configured to be coupled to the handle and to be inserted into the uterus of the patient, the sheath comprising at least three lumens, a first lumen for a scope assembly which scope assembly includes a camera and an illumination source, a second lumen to permit passage of the embryo transfer catheter therethrough, and a third fluid supply lumen for inflow and outflow of fluid to and from the uterus;
inserting the embryo transfer catheter into the visualization sheath;
inserting the visualization sheath into the uterus;
supplying fluid to the third fluid supply lumen to insufflate the uterus with fluid;
advancing the embryo transfer catheter into the uterus and positioning the tip of the catheter over an optimal location for embryo transfer;
removing all fluid from the uterus with negative pressure;
transferring an embryo into the uterus by depressing an injector on the embryo transfer catheter; and
removing the visualization sheath from the uterus.

13. The method of claim 12, further comprising illuminating a portion of the uterus to be imaged with a light source and imaging the uterus that is illuminated with the camera.

14. The method of claim 12, further comprising delivering approximately 1 cc to 10 cc of fluid to the uterus via the third fluid supply lumen.

15. The method of claim 12, wherein the handle comprises wireless connectivity to a SaaS application to permit capturing of patient data that details the visual embryo transfer method.

16. The method of claim 15, wherein the patient data is placed into a physician portal to become available for a physician office to access the patient data.

17-22. (canceled)

23. An embryo transfer management system comprising:
a handle assembly including a video display for displaying the interior of a uterus of a patient upon imaging of the uterus with a camera;
a holder that secures the handheld assembly at the top of the system;
a scope assembly comprising a camera and an illumination source;
an embryo transfer catheter which is configured to deliver an embryo to the uterus upon imaging of the uterus with the camera when the uterus is illuminated by the light source;
a programmable pump operably coupled to the embryo transfer catheter to provide a controlled release of an embryo into the uterus of the patient;
a visualization sheath which is configured to be coupled to the handle assembly and to be inserted into the uterus of a patient, the sheath comprising at least four lumens, a first lumen for the scope assembly, a second lumen to permit passage of the embryo transfer catheter therethrough, a third fluid supply lumen for inflow of fluid to the uterus, and a fourth lumen for removing fluid from the uterus therethrough; and
a peristaltic pump fluidly coupled to the third lumen and a vacuum pump fluidly coupled to the fourth fluid supply lumen to automatically provide fluid to the uterus via the third fluid supply lumen and to remove fluid from the uterus through the fourth lumen.

24-40. (canceled)

41. A kit for performing a visual embryo transfer procedure comprising:
a reusable handle assembly;
an embryo transfer catheter which is configured to deliver an embryo to the uterus;
a visualization sheath which is configured to be coupled to the handle assembly and to be inserted into the uterus of a patient, the sheath comprising at least three lumens, a first lumen for insertion of scope assembly into the uterus comprising a camera and a light source, a second lumen to permit passage of the embryo transfer catheter therethrough, and a third fluid supply lumen for flow of fluid to and/or from the uterus; and
an embryo transfer culture medium.

42. The kit of claim 41, further comprising at least one of: a syringe, an hCG wash fluid for the uterus, and an embryo transfer culture medium.

43-44. (canceled)

45. The kit of claim 41, wherein the visualization sheath comprises a fourth lumen for removing fluid from the uterus.
